(19) United States
(12) Patent Application Publication
(10) Pub. No.: US 2014/0228729 A1
(43) Pub. Date: Aug. 14, 2014

(54) Knuckle stretcher

(71) Applicant: James Edward Crews, Carrollton, GA (US)

(72) Inventor: James Edward Crews, Carrollton, GA (US)

(21) Appl. No.: 13/573,239

(22) Filed: Feb. 14, 2013

(51) Int. Cl. A61F 5/042

(52) U.S. Cl. CPC A61F 5/042
USPC 602/22

(57) ABSTRACT

A method and apparatus for stretching the knuckles at the base of the fingers to relieve pain in the hand. This is achieved through a molded polymer splint, worn on the center two fingers, provides spacing and rigidity between the finger bases at varying degrees of separation and compression. Product can be produced in various sizes to accommodate different hands. Product is a non-invasive, non-medical device to be used on a regular basis as needed by the wearer.

### BACKGROUND OF THE INVENTION

The present invention relates generally to support braces more particularly to a support brace worn on the hand, for carpal tunnel syndrome treatment and prevention, more in particular, a finger brace worn on the long finger and ring finger, that when worn, forces the hand in to a relaxed position while being comfortable to the wearer.

### DESCRIPTION OF THE PRIOR ART

The hand and wrist have a complex bone and muscle structure and is well known for a tendency to injure. Injuries result from overexertion, hyperextension, or extended periods of use where the hand and wrist are in unusual lateral or rotary positions such as when engaged in physical activity, operating computers or smart phones. Injuries also can result from intensive work with hands performing repetitive tasks. One in twenty people in the United States suffer from carpal tunnel syndrome.

Prior art treatment of this condition is to perform surgery to the medial nerve in the in the carpal tunnel or use wrist braces to relieve pain. Surgery can be successful for several years but the pain can return and may require additional surgery later. Surgery does not always relieve this pain, it is invasive, and permanently weakens the hand. Wrist braces are used to support the area where the median nerve underneath the transverse carpal ligament. Wrist braces do not stop numbness in the hands or arms because when the hand suffers from stress and pain, the fingers draw up in to a fist and circulation will not be adequate to stop numbness in the hand and arm and allow the hand to recover, and the condition persists and can worsen.

### SUMMARY OF THE INVENTION

It is an object of the with the present invention is to provide a hand brace for the prevention of carpal tunnel syndrome.

It is an object of the present invention to correct existing carpal tunnel syndrome.

It is also another object of the present invention to provide relief of numbness and pain in the hand and arms by gently forcing the fingers to stay in a relaxed position and stretching the metacarpophalangeal joint by the user wearing at the base of the middle finger and ring finger.

It is an object of the present invention to provide a hand brace that will prevent reoccurrence of carpal tunnel syndrome.

### BRIEF DESCRIPTION OF DRAWINGS

In The Drawings all are closely related

FIG. 1 is an elevated frontal view of the brace showing the shape of the outer enclosure.

FIG. 2 is an elevated side view of brace showing the shape of outer enclosure.

FIG. 3 is an plan view of brace showing the shape of outer enclosure with 2 perforations in center of apparatus.

FIG. 4 is a perspective view showing the perforations for the middle finger and ring finger.

### REFERENCE NUMERALS IN DRAWINGS

10 rounded edge
12 short side of enclosure
14 long side of enclosure
16 perforation

### DETAILED DESCRIPTION OF DRAWINGS

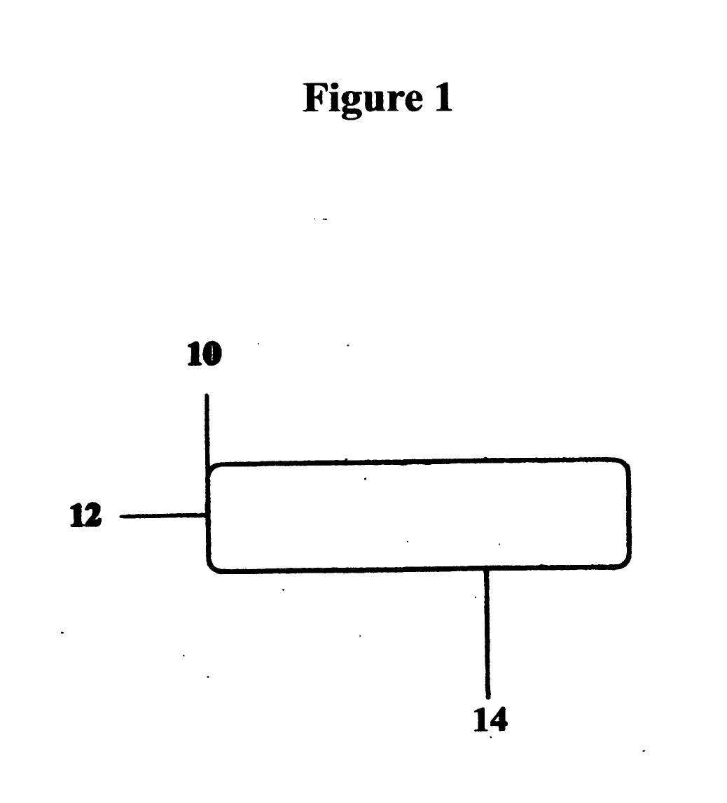

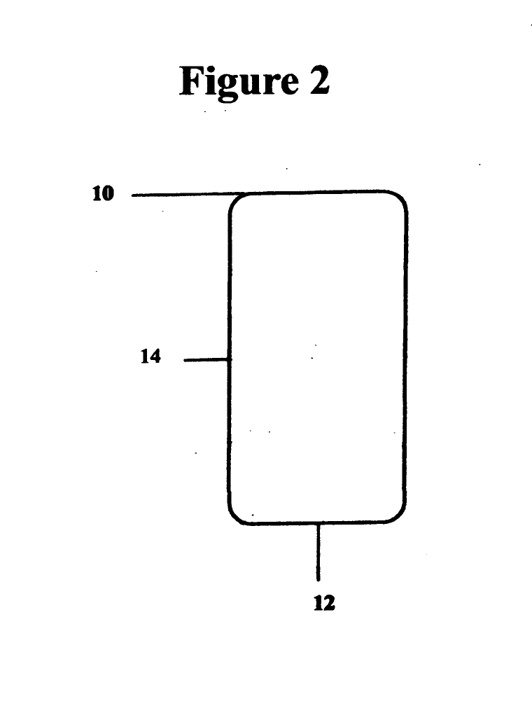

### FIGS. 1 and 2

The present invention is a brace to relieve pain in the hand and medial nerve, for use on either hand, fabricated from any flexible material, such as molded polymer. Moreover the edges 10 of the embodiment, are generally rounded to promote the comfort of the wearer when in a use condition. Illustrations 1 and 2 show elevated views of the exterior of the brace, when worn on the middle and ring fingers, including rounded edges 10, the short side 12, and the long side 14.

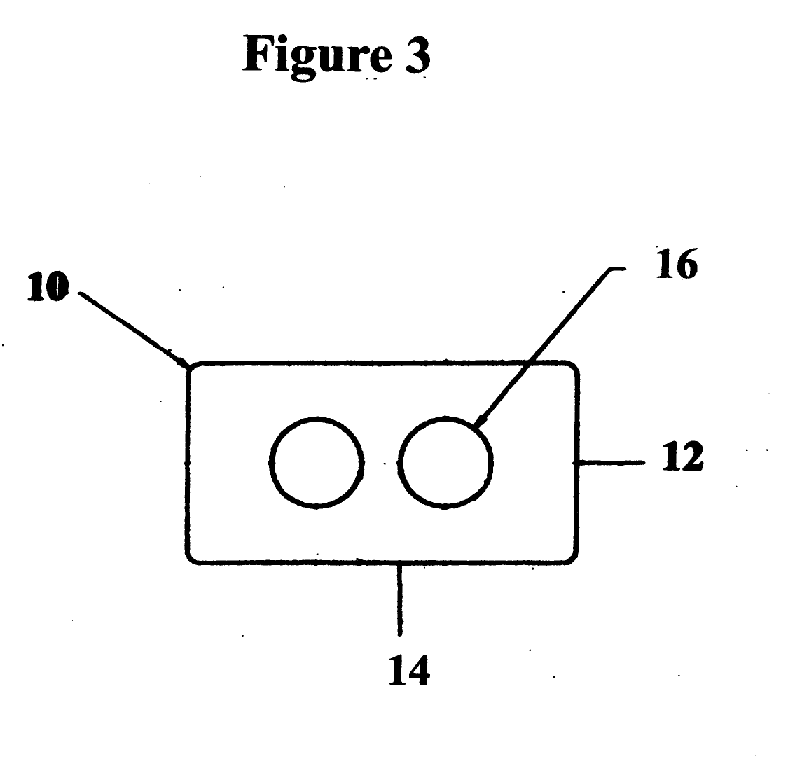

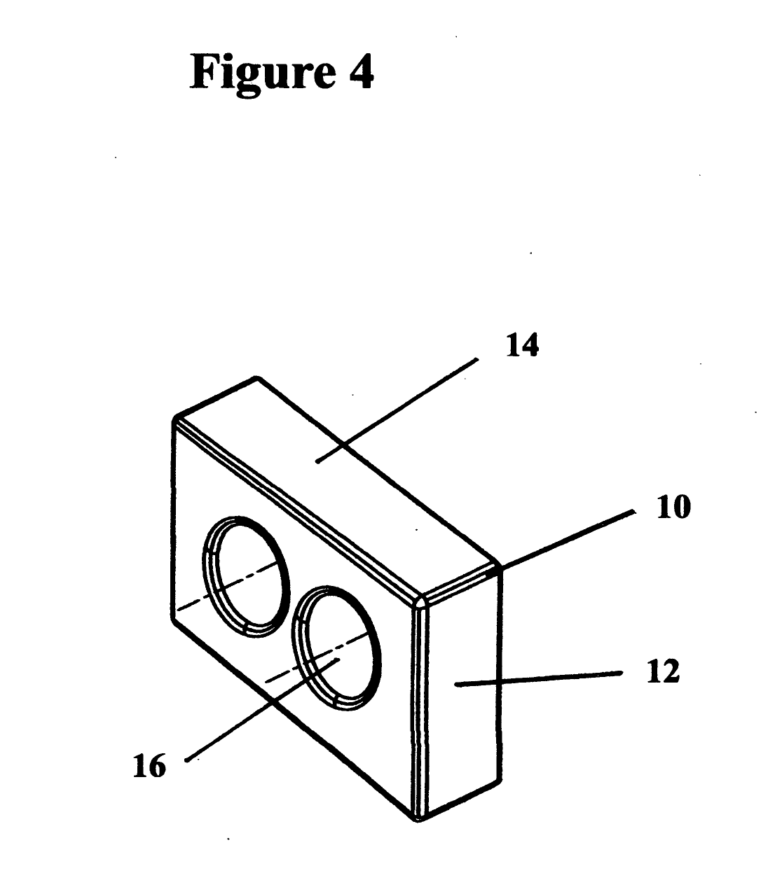

### FIGS. 3 and 4

Illustrate the spaced perforations 16 in the overall embodiment where the long finger and ring finger are placed inside the brace when worn by the user on either hand. Using the embodiment described will gently stretch the metacarpophalangeal joints and force the hand to relax from remaining in a clenched or fist position. Blood circulation in the hand greatly increases when the hand is relaxed and prevents numbness in the hand and arm and relieves pain in hand, medial nerve, and carpal tunnel area.

Rounded edges 10 short side 12 and long side 14 are shown from an plan view in FIG. 3 and perspective view in FIG. 4.

### OBJECTS AND ADVANTAGES

1 to provide a comfortable brace to prevent further damage to the hand from carpal tunnel syndrome.
2 to correct poor circulation in the hand and arms allowing the body to repair the existing carpal tunnel syndrome,
3 to restore use of hands.
4 to provide a comfortable hand brace that can be produced in many colors.
5 to stop numbness from occurring in the hand and arm.
6 to stop pain while using the hand.
7 to have an affordable alternative to surgery.
8 to force hand to relax and keep from remaining drawn in to a fist while sleeping.

Further objects and advantages are to provide a new brace that can be worn while sleeping that will prevent the hand and arm from becoming numb because of the hand being forced to relax, instead of being in a fist position reacting to pain or stress, and allowing the user to receive a better rest.

Still further objects and advantages will become apparent from a consideration of the ensuing description and drawings.

What is claimed is:

1a. A splint for the hand comprising:
Spaced perforations for stretching the metacarpophalangeal joint when worn at the base of the middle and ring fingers.
b A rectangular element encasing the perforations to maintain the spacing of the perforations so as to enable said perforations usable for stretching the metacarpophalangeal joints.
c A splint for the hand worn on the fingers.
